SEQUENCE LISTING (1 sequence)

SEQ ID NO: 1 - protein, 22 aa, Artificial Sequence
  Synthetic Peptide
PVLDLFRELLNELLEALKQKLK

(19) United States
(12) Patent Application Publication
(10) Pub. No.: US 2007/0197442 A1
(43) Pub. Date: Aug. 23, 2007

(54) Methods for the Treatment of Macular Degeneration and Related Eye Conditions

(76) Inventors: Milton L. Pressler, Ann Arbor, MI (US); William Sasiela, Montgomery Township, NJ (US)

Correspondence Address: PHARMACIA & UPJOHN, 7000 Portage Road, KZO-300-104, KALAMAZOO, MI, 49001, US

(21) Appl. No.: 11/676,739

(22) Filed: Feb. 20, 2007

Related U.S. Application Data
(60) Provisional application No. 60/775,140, filed on Feb. 21, 2006.

(51) Int. Cl. A61K 38/17; A61K 31/685

(52) U.S. Cl. 514/12; 514/78

(57) ABSTRACT

The invention provides a peptide-lipid complex for treating age-related macular degeneration and related conditions. The compositions and methods of the instant invention encompass a novel approach to the treatment of age-related macular degeneration and related conditions. Pharmaceutical compositions for this use are also provided.

### CROSS REFERENCE TO RELATED APPLICATIONS

This application claims the benefit of U.S. Provisional Patent Application No. 60/775,140, filed on Feb. 21, 2006, which is hereby incorporated in its entirety.

### 1. FIELD OF THE INVENTION

The present invention is directed to the fields of ophthalmology and cell biology of vision. Specifically, the present invention regards the treatment, amelioration or prevention of age-related macular degeneration (ARMD), including nonexudative (Dry ARMD) and exudative (Wet ARMD) forms. The present invention encompasses novel compositions and methods to treat ARMD and related eye disorders. In one embodiment, the method utilizes, or the composition comprises a discoidal peptide/phospholipid complex.

### 2. BACKGROUND OF THE INVENTION

Age-related macular degeneration (ARMD) is one of the leading causes of severe visual loss in the developed world (H. Taylor et al., Br. J. Ophthalmol., 2001; 85: 261-266; and M. VanNewkirk et al., Ophthalmology, 2000; 107: 1593-1600). In the early stages of the disease, before visual loss occurs from choroidal neovascularization, there is progressive accumulation of lipids in Bruch's membrane (D. Pauleikhoff et al., Ophthalmology, 1990; 97: 171-178; F. G. Holz, Arch. Ophthalmol., 1994; 112: 402-406; G. Sheraidah et al., Ophthalmology, 1993; 100: 47-51; and R. F. Spaide et al., Retina, 1999; 19: 141-147). Bruch's membrane lies at the critical juncture between the outer retina and its blood supply, the choriocapillaris. Progressive lipid deposition causes reduced hydraulic conductivity and macromolecular permeability in Bruch's membrane and thereby may impair retinal metabolism (D. J. Moore et al., Invest. Ophthalmol. Vis. Sci., 1995; 36: 1290-1297; D. Pauleikhoff et al., Ophthalmology, 1990; 97: 171-178; and C. Starita, C., Exp. Eye Res., 1996; 62: 565-572). After sufficient deposition of cholesterol and other lipids in Bruch's membrane, retinal pigmented epithelial cells (RPE) may respond by elaboration of angiogenic factors (e.g., VEGF, vEGE) that promote growth of new vessels from the choroid.

An open question is the pathogenesis of lipid deposition that ultimately triggers neovascularization. Interestingly, there are parallels between the lipid accumulation in Bruch's membrane found in ARMD and that observed in an animal model of atherosclerosis, the apolipoprotein E (apo E) null mice (Stefan Dithmar et al., Invest. Ophthalmol. Vis. Sci., 2000; 41: 2035-2042; and Mike Kliffen et al., Br. I. Ophthalmol., 2000; 84: 1415-1419). Immunohistochemistry on post-mortem eyes has demonstrated apo E in the basal aspect of the retinal pigmented epithelium (RPE) (D. H. Anderson et al., Am. S. Ophthalmol., 2001; 131(6): 767-781). Cultured RPE cells synthesize high levels of apo E mRNA, comparable to levels found in brain (D. H. Anderson et al., Am. J. Ophthalmol., 2001; 131(6): 767-781). Apolipoprotein E and apo E alleles may be a common denominator associated with several age-related degenerations, for example Alzheimer's disease and atherosclerosis. These associations have stimulated recent investigation of the potential role of apo E in ARMD. Several studies on apo E polymorphism in ARMD have been conducted to find linkages to specific alleles (F. Simonelli et al., Ophthalmic Res., 2001; 33: 325-328; C. C. Klayer et al., Am. J. Hum. Genet., July 1998; 63(1): 200-6; and E. H. Souied, Am. J. Ophthalmol., 1998; 125(3): 353-9). In contrast to Alzheimer's disease, the apo E-4 allele has been associated with reduced prevalence of ARMD. Apo E-2 allele is slightly increased in patients with AD. Further supporting a role in ARMD pathogenesis, apo E has been detected in drusens, the Bruch's membrane deposits that are the hallmark of ARMD (C. C. Klayer et al., Am. J. Hum. Genet., July 1998; 63(1): 200-6; and D. H. Anderson et al, Am. J. Ophthalmol., 2001; 131(6): 767-7).

While the role of apo E in ARMD is suggested but not established, this apolipoprotein has several functions that may affect the course of this disease. Apo E has anti-angiogenic (P. J. Browning et al., J. Exp Med., 1994; 180(5): 1949-54), anti-inflammatory (Micheal E. Kelly et al., Cellular Immunology, December 1994; Vol. 159, Issue 2: 124-139), and anti-oxidative effects (R. K. Tangirala et al., J. Biol. Chem., Jan. 5, 2001; 276(1): 261-6). These are all considered atheroprotective attributes of Apo E, but may also be important in protecting against progression of ARMD. While atheroprotective effects of apo E were initially thought to stem from its effects on plasma lipid levels, local effects on vascular macrophages are probably equally important. Thus, selective enhanced expression of macrophage apo E in the arterial wall reduces atherosclerosis in spite of hyperlipidemia (H. Shimano et al., J. Clin. Invest., 95: 469-476; S. Bellosta, et al., J. Clin. Invest., 1995; 96: 2170; and A. H. Hasty et al., Circulation, 1999; 99: 2571-2576). Conversely, reduction of Apo E levels by reconstitution of apo E null macrophages into C57BL/6 wild type mice fosters the development of atherosclerosis (Fazio et al., 1994-need citation).

Atheroprotective effects of arterial apo E expression are thought to derive in part from facilitation of reverse cholesterol transport (T. Mazzone and C. Reardon, Circulation, 1992; 86(Suppl. 1): 1-2; and C. Y. Lin et al., J. Lipid Res., 1999; 40: 1618-1626). The mechanisms by which apo E facilitates reverse cholesterol transport are incompletely understood. Apo E expression increases cholesterol efflux to HDL3 in J774 macrophages (T. Mazzone and C. Reardon, J. Lipid Res., 1994; 35: 1345-1353) and lipid free apolipoprotein A1 (C. Langer et al., J. Mol. Med., 2000; 78: 217-227). Cell surface apo E is also hypothesized to induce efflux from the plasma membrane (C. Y. Lin et al., J. Lipid Res., 1999; 40: 1618-1626).

Cholesterol transport may be important in the pathogenesis of ARMD because of lipid efflux from PE into Bruch's membrane. Very much like intimal macrophages, RPE cells progressively accumulate lipid deposits throughout life; however, unlike vessel wall macrophages, the source of RPE lipid is thought to be retinal photoreceptor outer segments (POS) (C. J. Kennedy et al., Eye, 1995; 9: 262-274). Every day, each RPE cell phagocytoses and degrades more than one thousand POS via lysosomal enzymes. These POS are enriched in phospholipid and contain the photoreactive pigment, rhodopsin. Incompletely digested POS accumulate as lipofuscin in RPE. By age 80, approximately 20% of RPE cell volume is occupied by lipofuscin (L. Feeney-Burns et al., Invest. Ophthalmol. Vis. Sci., 1984; 25: 195-200).

Analysis of Bruch's membrane lipid reveals an age-related accumulation of phospholipid, triglyceride, cholesterol, and cholesterol ester (F. G. Holz, Arch. Ophthalmol., 1994; 112: 402-406; and C. A. Curcio et al., Invest. Ophthalmol. Vis. Sci., 2001; 42: 265). The origin of these lipids also is thought to derive principally from POS rather than from the circulation (F. G. Holz, Arch. Ophthalmol., 1994; 112: 402-406; and R. F. Spaide et al., Retina, 1999; 19:141-147). POS lipids are hypothesized to efflux from the RPE into Bruch's membrane. Although cholesterol ester deposition in Bruch's suggests contribution from plasma lipids, biochemical analysis of these ethers suggests etherification of intracellular cholesterol by RPE cell derived ACAT (C. A. Curcio, et al., ARVO Abstracts, 2002). While trafficking of lipids from the retina to RPE cells has been studied extensively, mechanisms of lipid efflux from RPE to Bruch's membrane are not well understood. Furthermore, from a pathogenic standpoint, regulation of lipid efflux into Bruch's membrane may be important in determining the rate of lipid-induced thickening that occurs in aging.

Reverse cholesterol transport in macrophages is regulated by nuclear hormone receptor ligands via their effects on ABCA-1 and apo E expression. Liver X receptor (LXR) and/or retinoid X receptor (RXR) ligands increase levels of these transporters and increase reverse cholesterol transport in macrophages (P. A. Mak et al., J. Biol. Chem., Aug. 30, 2002; 277(35): 31900-8; and B. A. Laffitte et al., Proc. Natl. Acad. Sci. USA, Jan. 16, 2001; 98(2): 507-12). Thyroid hormone has also been demonstrated to increase expression of apo E three fold in HepG2 cells (B. A. Laffitte et al., Eur. J. Biochem., Sep. 1, 1994; 224(2): 463-71).

In atherosclerosis (AS), lipids accumulate in the extracellular matrix and within phagocytic cells, primarily macrophages. Mechanisms of lipid metabolism in AS have been investigated in detail. Similar investigations into lipid processing by RPE and subsequent lipid efflux into BM and the circulation have not been conducted with the same depth as those for AS. As a consequence, potential therapeutic approaches to dry ARMD are wanting.

M. Navab et al., Curr. Opin. Investig. Drugs, 2003; 4(9): 1100-4, describe Apo A-I mimetic peptides comprising D-amino acids for oral delivery for the treatment of atherosclerosis.

U.S. Patent Application Publication US 2002/0142953 relates to human compositions encoding apolipoproteins that are related to lipid metabolism and cardiovascular disease.

R. F. Mullins, et al., FASEB J, May 2000 May; 14(7): 835-46, describe compositional similarity between drusen and other extracellular deposits, including atherosclerotic plaques. Specifically, vitronectin, amyloid P, Apo E and lipids are among the constituents shared in common. More specifically, apolipoprotein E is identified in retinal pigmented epithelium.

E. Friedman, Amer. J. Ophthalm., 2000; 130(5): 658-663, reviews the role of atherosclerosis in the pathogenesis of ARMD. Specifically, the review mentions targeting the angiogenesis pathway for treating the neovascular form of ARMD, such as the member VEGF. It is noted that interfering with the upregulation or action of angiogenic agents may prove helpful for choroidal neovascularization, and, in alternative embodiments, statins may be useful for lowering the risk of

D. H. Anderson et al, Am. J. Ophthalmol., 2001; 131(6): 767-781, report that apolipoprotein E protein is found in the same location as drusen, likely originating from the retinal pigmented epithelium.

U.S. Pat. No. 6,071,924 regards inhibition of proliferation of retinal pigmented epithelium by contacting RPE cells with a retinoic acid receptor agonist, except for retinoic acid, preferably thereby inhibiting AP-1-dependent gene expression. In specific embodiments, an AP1 antagonist is delivered to a subject in need thereof for inhibition of proliferation of retinal pigment epithelium or a disease associated therewith.

U.S. patent No. is directed to inhibition of choroidal neovascularization by contacting RPE cells with an AP-1 antagonist.

U.S. Pat. No. 5,824,685 regards amelioration of proliferative vitreoretinopathy or traction retinal detachment by contacting RPE cells with a retinoic acid receptor selected from ethyl-6-[2-(4,4-dimethylthiochroman-6-yl)ethynyl]nicotinate, 6-[2-(4,4-dimethylchroman-6-yl)ethynyl]nicotinic acid, and p-[(E)-2-(5,6,7,8-tetrahydro-5,5,8,8-tetramethyl-2-naphthyl)propenyl]-benzoic acid.

U.S. Pat. No. 6,372,753 addresses inhibition of an ocular disease resulting from proliferation of retinal pigmented epithelium by providing at least one AP-1 antagonist and at least one retinoic acid receptor (RAR) agonist, except for retinoic acid.

WO 01/58494 is directed to treating or preventing an ocular disease, such as age-related macular degeneration, by contacting an ocular cell with an expression vector comprising a nucleic acid sequence encoding an inhibitor of angiogenesis and a neurotrophic agent. In specific embodiments, the inhibitor of angiogenesis and the neurotrophic agent are one and the same, such as pigment epithelium-derived factor (PEDF).

WO 02/13812 regards the use of an insulin-sensitizing agent, preferably peroxisome proliferator-activated receptor y (PPAR y) agonists, for the treatment of an inflammatory disease, such as an ophthalmic disease.

WO 00/52479 addresses diagnosing, treating, and preventing drusen-associated disorders (any disorder which involves drusen formation), including ARMD. In specific embodiments, there are methods related to providing an effective amount of an agent that inhibits immune cell proliferation or differentiation, such as antagonists of the cytokine, tumor necrosis factor (TNF)-alpha.

WO 2004/098506 and WO 2004/0266663 describe the treatment of age-related macular degeneration using regulation of pathogenic mechanisms similar to atherosclerosis. In specific embodiments, reverse cholesterol transport components, such as transporters and HDL fractions, are utilized as diagnostic and therapeutic targets for age-related macular degeneration. In a further specific embodiment, the lipid content of the retinal pigmented epithelium, and/or Bruch's membrane is reduced by delivering Apolipoprotein AI, particularly a mimetic peptide.

Jorge G. Arroyo in the “Age-related macular degeneration-I and II,” UpToDate (on-line service) (last modified Sep. 19, 2005), provides background information on age-related macular degeneration, including epidemiology, etiology/risk factors, clinical presentation, diagnosis, treatment and prevention.

J. L. Duncan in “Mouse models may provide new insight into the relation between cholesterol and age related macular degeneration,” Downloaded from bjo.bmjjournals.com on 5 Dec. 2005, descries that the findings in LDL receptor deficient mice may provide insight into the mechanism of early ARMD.

M. Rudolf et al. in “Increased expression of vascular endothelial growth factor associated with accumulation of lipids in Bruch's membrane of LD receptor knockout mice,” Downloaded from bjo.bmjournals.com on 5 Dec. 2005, describe that LDL receptor deficient mice exhibit an accumulation of lipid particles in Bruch's membrane which is further increased after fat intake.

According to the preliminary program for the 20% Macula Society meeting (www.maculasociety.org), one of the abstracts at the 29^{th} Annual Macula Society Meeting (which will take place Feb. 22-25, 2006) (North San Diego, Calif.), is titled “An Epidemiologic Study of Age-Related Macular Degeneration (AMD) in Carriers of the Apolipoprotein A-I-Milano Mutation: A Novel Treatment Approach to AMD,” by Jorge G. Arroyo.

S. Nissen et al., JAMA, November 2003; 290: 2292-2300 discloses a study in which atheroma volume is reduced after treatment with recombinant apoA-I Milano-POPC complex.

ApoA-I agonist peptides, typically 15-30 amino acids in length, mimic the activities of apoA-I. In particular, the apoA-I agonists bind lipids, form amphipathic helices (in the presence of lipids), form HDL-like complexes and may activate LCAT (e.g., U.S. Pat. No. 6,004,925 (issued Dec. 21, 1999) and U.S. Pat. No. 6,630,450 (issued Oct. 7, 2003), the contents of which are hereby incorporated herein in its entirety).

Thus, there is a need for a new approach for the treatment, amelioration or prevention of age-related macular degeneration by reducing the size and lipid content of drusens and other pathological lesions of the retina and ocular tissue, such as Bruch's membrane.

### 3. SUMMARY OF THE INVENTION

The invention encompasses methods and compositions for the treatment of age-related macular degeneration or a related disorder in a mammal using a peptide/phospholipid complex. In one embodiment, the peptide/phospholipid complex, comprises an apoA-I agonist peptide, termed ESP24218 (SEQ ID NO:1), sphingomyelin and 1,2-dipalmitoyl-sn-glycero-3-phosphocholine (DPPC), which complex is referred to as ETC-642.

In one aspect, the present invention provides compositions and methods for the treatment of age-related macular degeneration, its various forms (e.g., wet, dry; early, late) or related conditions by administering a composition comprising the peptide/phospholipids complex, referred to as ETC-642, to a subject in need thereof.

Methods, compositions and dosage regimens are provided herein, and are believed to encompass safe, effective and non-surgical treatments, without being limited by theory, that rapidly promote cholesterol efflux and mobilization from lipidic plaques in the retina or Bruch's membrane (e.g. drusens), which thereby confer benefit in terms of improvement of visual acuity, prevention of loss of visual acuity, or improvement or prevention of macular degeneration that leads to impaired visual acuity. The mechanism of action encompasses improved blood flow or perfusion of the retina or choroid plexus that directly or indirectly confers improvement in visual acuity, or prevention of loss of visual acuity, or confers amelioration of lesions that promote neovascularization of the retina that reduce visual acuity.

In one aspect, the present invention provides administering to a mammal in need thereof a peptide/phospholipid complex comprising ESP24218 (SEQ ID NO:1), sphingomyelin and 1,2-dipalmitoyl-sn-glycero-3-phosphocholine (DPPC). In certain embodiments, the peptide/phospholipid complex consists essentially of ESP24218 (SEQ ID NO:1), sphingomyelin and 1,2-dipalmitoyl-sn-glycero-3-phosphocholine (DPPC). In certain embodiments, the peptide/phospholipid complex consists essentially of ESP24218 (SEQ ID NO:1), sphingomyelin and DPPC in approximately equal proportions.

Typically, the peptide/phospholipid complex is administered as a sterile liquid pharmaceutical formulation.

In certain embodiments, the peptide/phospholipid complex is administered to a human at a dose of between about 1 mg/kg to about 20 mg/kg. In some embodiments, the peptide/phospholipid complex is administered to a human at a dose of about 5 mg/kg, about 10 mg/kg or about 15 mg/kg. It will be apparent to those of skill in the art that the doses described herein are based on the human patient's body mass.

The pharmaceutical formulation provided herein comprises a peptide/phospholipid complex in a suitable pH, osmolality, tonicity, purity and sterility to allow safe administration to a subject. The pharmaceutical formulation generally comprises an excipient. For example, in certain embodiments, a sterile liquid pharmaceutical formulation consists essentially of peptide/phospholipids complex and an aqueous buffer.

### 4. DETAILED DESCRIPTION OF THE INVENTION

4.1 Definitions

As used herein, the following terms shall have the following meaning:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “age-related macular degeneration” as used herein refers to macular degeneration, both wet and dry forms, early or late stage, in an individual over the age of about 50. In one specific embodiment, it is associated with destruction and loss of the photoreceptors in the macula region of the retina resulting in decreased central vision and, in advanced cases, legal blindness.

The term “Bruch's membrane” as used herein refers to a five-layered structure separating the choriocapillaris from the retinal pigmented epithelium, RPE.

The term “increase lipid efflux” or “increasing lipid efflux” as used herein refers to an increased level and/or rate of lipid efflux, promoting lipid efflux, enhancing lipid efflux, facilitating lipid efflux, upregulating lipid efflux, improving lipid efflux, and/or augmenting lipid efflux. In one embodiment, the efflux comprises efflux of phospholipid, triglyceride, cholesterol, and/or cholesterol ester.

The term “macula” as used herein refers to the light-sensing cells or photoreceptors of the central region of the retina.

The term “macular degeneration” as used herein refers to deterioration of the central portion of the retina, the macula.

The term “subject” refers to an animal such as a mammal, including but not limited to a primate (e.g., a human), a dog, a cat, a rabbit, a rat, a mouse and the like. In specific embodiments, the subject is a human.

The terms “treat”, “treating” or “treatment” refer to alleviating, reducing, abrogating, or otherwise modulating a disease, disorder and/or one or more symptoms thereof, that is a therapeutic effect on an existing condition

The term “therapeutically effective amount” refers to that amount of an active ingredient sufficient to improve one or more of the symptoms of the condition or disorder being treated as compared to those symptoms that occur without treatment. The improvement may be temporary or permanent.

The term “prophylactically effective amount” refers to that amount of an active ingredient sufficient to result in the prevention, onset or recurrence of one or more symptoms of the condition or disorder.

The terms “prevent”, “preventing” or “prevention” refer to barring, or reducing the risk of, a subject from acquiring a disease, disorder and/or symptoms thereof.

The term “pharmaceutical formulation” refers to a composition comprising either an active ingredient and a suitable diluent, carrier, vehicle, or excipients suitable for administration to a subject. The pharmaceutical formulation or composition will comprise ETC-642. The terms “composition” and “formulation” are used interchangeably herein. The term is also meant to encompass situations wherein the components of the combination therapy are in the same or separate formulations. This term includes, but is not limited to oral, parenteral, intraocular, mucosal and topical compositions as described below.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

The term “label” refers to a display of written, printed or graphic matter upon the immediate container of an article, for example, the written material displayed on a vial containing a pharmaceutically active agent.

The term “labeling” refers to all labels and other written, printed or graphic matter upon any article or any of its containers or wrappers or accompanying such article, for example, a package insert, instructional videotapes or instructional DVDs accompanying or associated with a container of a pharmaceutically active agent.

4.2. Preparation of Peptide/Phospholipid Complex and Pharmaceutical Formulations

In the methods of the invention, the peptide/phospholipid complex provided comprises ESP242118 (SEQ ID NO:1) and one or more phospholipids. In certain embodiments, the peptide/phospholipid complex provided comprises ESP24218 (SEQ ID NO:1), sphingomyelin and 1,2-dipalmitoyl-sn-glycero-3-phosphocholine (DPPC). In certain embodiments, the peptide/phospholipid complex consists essentially of ESP24218 (SEQ ID NO:1), sphingomyelin and DPPC. In other embodiments, the peptide/phospholipid complex consists of ESP24218 (SEQ ID NO:1), sphingomyelin and DPPC.

For example, the exemplary peptide/phospholipid complex ETC-642 is composed of ESP24218 (SEQ ID NO:1), a synthetic amphipathic peptide, and two naturally occurring phospholipids, sphinogmyelin and 1,2-dipalmitoyl-sn-glycero-3-phosphocholine (DPPC). ESP24218 (SEQ ID NO:1), sphingomyelin and DPPC are not individually active pharmaceutical ingredients or drug substances in ETC-642. ESP24218 (SEQ ID NO:1), sphingomyelin and DPPC are raw materials or components used to produce the ETC-642 drug substance. ETC-642 is believed to be a discoidal peptide/phospholipid complex.

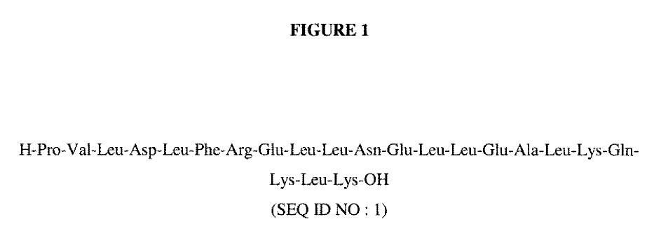

ESP24218 (SEQ ID NO:1), the peptide component of the peptide/phospholipid complex, is a 22 amino acid peptide having a helical structure and amphipathic properties designed based on the helical repeats of apoA-I. In the presence of lipids, this peptide approaches 100% of the activity of native apoA-I in in vitro LCAT activation. The peptide can be prepared by chemical synthesis using any technique known to those of skill in the art. The peptide can be prepared by the synthetic techniques described in U.S. Pat. No. 6,004,925, incorporated herein by reference in its entirety for all purposes. Typically, ESP24218 (SEQ ID NO:1) is produced by a stepwise chemical synthesis on a solid support. In preferable embodiments, all amino acids in the peptide are natural L-α-amino acids in configuration. Generally, the amino and carboxyl termini of the peptide are free. The amino acid sequence of ESP24218 (SEQ ID NO:1) is shown in FIG. 1, which is as follows: H-Pro-Val-Leu-Asp-Leu-Phe-Arg-Glu-Leu-Leu-Asn-Glu-Leu-Leu-Glu-Ala-Leu-Lys-Gln-Lys-Leu-Lys-OH. The phospholipid can be obtained from any source known to those of skill in the art. For example, the phospholipid can be obtained from commercial sources, natural sources or by synthetic or semi-synthetic means known to those of skill in the art (see, e.g., Mel'nichuk et al. Ukr. Biokhim. Zh. 59(6):75-7 (1987); Mel'nichuk et al., Ukr. Biokhim. Zh. 59(5):66-70 (1987); Ramesh et al., J. Am. Oil Chem. Soc. 56(5):585-7 (1979); Patel and Sparrow, J. Chromatogr. 150(2):542-7 (1978); Kaduce et al. J. Lipid Res. 24(10):1398-403 (1983); Schlueter et al., Org. Lett. 5(3):255-7 (2003), Tsuji et al., Nippon Yakurigaku Zasshi 120(1):67P-69P (2002)).

The phospholipid can be any phospholipid known to those of skill in the art. For example, the phospholipid can be a small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-paimitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipaimitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine. The phospholipid can also be a derivative or analogue of any of the above phospholipids. The phospholipid, dipalmitoylphosphatidylcholine (DPPC), may also be referred to as 1,2-dipalmitoyl-sn-glycero-3-phosphocholine (DPPC).

In certain embodiments, the lipid components of the peptide/phospholipid complex are sphingomyelin and 1,2-dipalmitoyl-sn-glycero-3-phosphocholine (DPPC). Sphingomyelin is a naturally occurring phospholipid present in biological membranes as well as human HDL, specifically in pre-βHDL. Sphingomyelin is characterized by its backbone, sphingosine, an amino alcohol that contains a long unsaturated hydrocarbon chain. Sphingomyelin may be prepared by extracting it from animal sources or chemically synthesized according to standard techniques well-known to those of skill in the art. Sphingomyelin can also be purchased from commercial sources, such as Lipoid GmbH (Ludwigshafen, Germany).

DPPC is a naturally occurring phospholipid consisting of a glycerol backbone and two palmitic acid chains. DPPC may be prepared by purifying it from a natural source or by chemical synthesis according to standard techniques well-known to those of skill in the art. Typically, DPPC is produced by chemical synthesis. DPPC can be purchased from commercial sources, such as Avanti Polar Lipids, Inc. (Alabaster Ala.).

Generally, the peptide/phospholipid complex is prepared by co-lyophilization of the peptide and lipid components as described, for example, in U.S. Pat. Nos. 6,287,590, 6,455,088, 6,004,925 and 6,376,464, the contents of which are each incorporated herein by reference in their entirety for all purposes. For example, the lyophilized peptide/lipid powder can be rehydrated for administration into a patient. Other methods of preparing peptide/phospholipid complexes will be apparent to those of skill in the art.

In one embodiment, the peptide/phospholipid complex can be in solution with an appropriate pharmaceutical diluent. In another embodiment, freeze-dried or lyophilized preparations of peptide/phospholipid complex can be hydrated or reconstituted with an appropriate pharmaceutical diluent prior to administration. In another embodiment, the peptide/phospholipid complex can be frozen preparations that are thawed until a homogenous solution is achieved prior to administration to a subject in need thereof.

The peptide/phospholipid complex can be administered in the form of a pharmaceutical formulation. A pharmaceutical formulation, as described herein, includes the addition of, for example, an acceptable diluent, excipient, vehicle or carrier. As is known in the art, the addition of one or more diluents, excipients, vehicle or carriers renders a formulation suitable for administration to a subject and can bestow other favorable properties such as extended shelf life.

In certain embodiments, the peptide/phospholipid complex is administered as a sterile liquid pharmaceutical formulation.

The pharmaceutical formulations typically comprise a pharmaceutically acceptable carrier or vehicle. Many pharmaceutically acceptable carriers or vehicles can be employed. In certain embodiments, the pharmaceutical formulation consists essentially of sterile bicarbonate saline and peptide/phospholipid complex in a solution. One advantage of sterile bicarbonate saline, for example, is it that effectively hydrates the lyophilized peptide/lipid powder to form dispersed peptide/phospholipid complexes in solution which can then be directly administered into a human.

In other embodiments, normal saline is employed as the pharmaceutical carrier or vehicle.

Other suitable carriers or vehicles include dextrose, glucose, trehalose, sucrose, sterile water, buffered water, 0.45% saline (half Normal saline), and 0.3% glycine, and can further include glycoproteins for enhanced stability, such as albumin. These formulations can be sterilized by conventional, well known sterilization techniques. The resulting aqueous solutions can be packaged for use or filtered under aseptic conditions and lyophilized (freeze-dried). The lyophilized preparation can then be combined with a sterile aqueous solution prior to administration.

The pharmaceutical formulations can also contain pharmaceutically acceptable excipients as required to approximate physiological conditions, such as pH adjusting and buffering agents, and tonicity adjusting agents, for example, sodium acetate, sodium lactate, sodium chloride, potassium chloride, and calcium chloride. Antibacterial agents, for example, phenol, benzalkonium chloride or benzethonium chloride, can be added to maintain sterility of a product, especially pharmaceutical formulations intended for multi-dose parenteral use. Suspending, stabilizing and/or dispersing agents can also be used in the formulations of the invention.

The pharmaceutical formulations can be in a variety of forms suitable for any route of administration which include, but are not limited to parenteral, intraocular, subcutaneous, transdermal, transmucosal and rectal administration.

In one embodiment, the composition is formulated in accordance with routine procedures as a pharmaceutical composition adapted for parenteral administration.

Parenteral administration refers to any route of administration that is not through the alimentary canal, including, but not limited to, injectable administration (i.e., intravenous, intraarterial, intramuscular, intradermal, intraocular, and the like as described herein) (see generally, Remington's Pharmaceutical Sciences, 18^{th} Edition, Gennaro et al., eds., Mack Printing Company, Easton, Pa., 1990).

The pharmaceutical formulation of the present invention can be in a form suitable for an ocular route of administration, which includes, but is not limited to an intravitreal, intraocular (intracameral), subconjunctival, sub-Tenon's, retrobulbar injection and a topical application. In one embodiment, the pharmaceutical formulation is administered as an intravitreal injection. The injectable pharmaceutical formulation of the present invention is ophtalmically acceptable, i.e., it is appropriate for administration directly into the eye including aqueous and vitreous humors.

Injectable pharmaceutical formulations can be sterile suspensions, solutions or emulsions in aqueous or oily vehicles. The formulations for injection can be presented in unit dosage form, e.g., in ampoules or in multidose containers, and can comprise added preservatives.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Compounds that increase the solubility of the active ingredient disclosed herein can also be incorporated into the parenteral dosage forms of the invention.

In another embodiment, the pharmaceutical formulation can be provided in powder form for reconstitution with a suitable vehicle, including but not limited to sterile pyrogen free water, saline or dextrose before use. For example, ESP24218 (SEQ ID NO:1) can be co-lyophilized with sphingomyelin and DPPC. In another embodiment, the pharmaceutical formulations can be supplied in unit dosage forms and reconstituted prior to use.

Targeting of ocular tissues may be accomplished in any one of a variety of ways. Injection into the aqueous or vitreous humor of the eye is one means. Directly injecting the ETC-642 into the proximity of the RPE or Bruch's membrane provides targeting of the complex with some forms of ARMD. In one embodiment, the complex is administered via intra-ocular sustained delivery (such as Vitrasert® or Envision® by Bausch and Lomb). In another embodiment, the compound is delivered by posterior suborbital injection.

In certain embodiments, the pharmaceutical formulation can be a unit-of-use package. As is known to those of skill in the art, a unit-of-use package is a convenient, prescription size, patient ready unit labeled for direct distribution by health care providers. A unit-of-use package contains a pharmaceutical formulation in an amount necessary for a typical treatment interval and duration for a given indication. The methods of the invention provide for a unit-of-use package of a pharmaceutical formulation comprising, for example, peptide/phospholipid complex in an amount sufficient to treat an average sized adult male or female with 20 mg (peptide equivalent)/kg intravenously once weekly for 4 weeks. Thus a unit of use package as described above would have four doses of peptide/phospholipid complex, each preferably in a lyophilized form in a vial which is hydrated, for example, with a sterile bicarbonate solution in order to be administered to a human.

The pharmaceutical formulations can be labeled and have accompanying labeling to identify the formulation contained therein and other information useful to health care providers and subjects in the treatment and prevention of age-related macular degeneration, including, but not limited to, instructions for use, dose, dosing interval, duration, indication, contraindications, warnings, precautions, handling and storage instructions and the like.

In further embodiments, the present invention provides kits for treating or preventing age-related macular degeneration. The kits comprise one or more effective doses of peptide/phospholipid complex or pharmaceutical formulations thereof along with a label or labeling with instructions on using the peptide/phospholipid complex or pharmaceutical formulations thereof to treat or prevent age-related macular degeneration according to the methods of the invention. In certain embodiments, the kits can comprise components useful for carrying out the methods such as devices for delivering the peptide/phospholipid complex or pharmaceutical formulations thereof and components for the safe disposal of these devices, e.g. a sharps container. In certain embodiments, the kits can comprise peptide/phospholipid complex or pharmaceutical formulations thereof in a pre-filled syringes, unit-dose or unit-of-use packages.

4.3 Use of ETC-642 for Treating Age-Related Macular Degeneration (ARMD)

The doses of ETC-642 sufficient to treat age-related macular degeneration or related eye disorder in a mammal in need thereof are described herein. Doses useful for treatment of age-related macular degeneration include doses up to about 20 mg/kg of the ETC-642 administered intravenously to a subject in need thereof. In certain embodiments, the compositions and methods comprise administration of the ETC-642 at a dose of about 1 mg/kg to about 20 mg/kg of a subject's body weight. In particular embodiments, the compositions and methods comprise intravenous administration of ETC-642 at a dose of about 5 mg/kg to about 10 mg/kg. In other embodiments, the composition and methods comprise intra-ocular administration of ETC-642 at a dose of about 0.2 to about 1 mg/kg.

It is understood by those of skill in the art that the actual dose of ETC-642 according to the present invention can vary with the route of administration, height, weight, age, severity of illness of the subject, the presence of concomitant medical conditions and the like. For example, an elderly subject with compromised renal or liver function can be treated with a dose of the ETC-642 that is at the lower range of about 1 mg/kg dose (e.g., 0.8 mg/kg or 0.9 mg/kg) as part of the therapy. A subject with severe cardiovascular disease or related disorders that is obese with good renal and liver function can be treated with a dose of the ETC-642 that is, for example, at the upper range of about 20 mg/kg dose as part of the therapy. These doses achieve a range of circulating concentrations that include the effective dose with an acceptable risk-to-benefit profile.

The dose of ETC-642 can vary over the duration of treatment. For example, a subject can be treated with a higher dose of the pharmaceutical formulation of the ETC-642, intravenously once weekly for 3 weeks and then treated with a lower dose of the ETC-642 once every four months or once per year for the lifetime of the subject. Such intermittent doses can be administered to maintain a reduced size and number of drusens in Bruch's membrane. Intermittent doses during the lifetime of the subject to maintain a reduced volume of lipids in the drusens are within the scope of the present invention.

In certain embodiments, a single high dose of the ETC-642 is administered intravenously to the subject. In other embodiments, one or more high doses of the ETC-642 are administered to the subject followed by one or more of the same or lower doses of the ETC-642. Additionally, the opposite regimen may be used comprising administration of one or more low doses of the ETC-642 followed by one or more of the same or higher doses of the ETC-642. For more advanced stages of macular degeneration (e.g., wet forms), the high dose in certain embodiments is delivered first.

In certain embodiments, the ETC-642 is administered as an intravenous infusion. In certain embodiments, the compositions and methods comprise administration as an intravenous push infusion. In certain embodiments, the ETC-642 is administered intravenously by intravenous push infusion over a short time period, such as up to 5 minutes, for example, 2-5 minutes. In certain embodiments, administration of the ETC-642 comprises a continuous intravenous infusion. In certain embodiments, ETC-642 is administered by continuous intravenous infusion over a period of time, for example, about 1 hour to 3 hours, preferably, about 30 minutes to 120 minutes. Continuous intravenous infusions can be administered with the aid of an infusion pump or device. In certain embodiments, administration of the ETC-642 can be a combination of continuous intravenous infusions and intravenous push infusions (“bolus doses”). The bolus doses can be administered before, after or during the continuous infusion.

The compositions and methods provide for intravenous infusion of the ETC-642. Any suitable blood vessel can be used for infusion, including peripheral vessels, such as the vessels in the antecubital fossa of the arm or a central line into the major veins of the chest. In certain embodiments, the pharmaceutical formulation is infused into the cephalic or median cubital vessel at the antecubital fossa in the arm of a subject.

4.4. Timing of Administration of ETC-642

In certain embodiments, the methods for the treatment of age-related macular degeneration or related conditions comprise administering the ETC-642 about every week, about every other week, about every 4, 5, 6, 7, 8 to 10 or 11 to 14 days to a subject in need thereof. In one embodiment, the ETC-642 can be administered about every 7 days. In certain embodiments, administration of the ETC-642 can be a one-time administration every six months or every year. In certain embodiments, administration can continue for about 1 week, about 2 weeks, about 3 weeks, about 4 weeks, about 5 weeks, about 6 weeks, about 7-12 weeks, about 13-24 weeks or about 25-52 weeks. In certain embodiments, administration of the ETC-642 is about every 7 days for about 5 weeks. In certain embodiments, administration can be intermittent after, for example, about 5 weeks. For example, a subject can be treated once a week for about 5 weeks and then treated about 3 to about 4 times over the following year. In certain embodiments, the pharmaceutical formulations described herein can be administered to the subject intermittently to maintain a reduced size and number of drusens in Bruch's membrane. For example, the appropriate dose of ETC-642 can be administered about every 10 days for about 7 weeks and then administered, for example, about 26 weeks later or about 52 weeks later.

4.5 Patients and Diseases to be Treated by the Therapy

The invention provides novel compositions and methods to treat age-related macular degeneration or related disorders or forms (wet, dry, early, late).

In some embodiments, the compositions and methods of the present invention are to treat age-related macular degeneration or related disorders in subjects with signs or symptoms thereof. Age-related macular degeneration (ARMD) is a clinical diagnosis based upon the presence of visual disturbance and characteristic findings on dilated eye examination. In patients with dry type of ARMD, drusens are visible on dilated eye examination. In such patients, round or oval patches of geographic atrophy of the retina may be evident as areas of depigmentation, increased pigmentation may be seen with RPE pigmentary mottling. In patients with wet type of ARMD, dilated examination may reveal subretinal fluid, hemorrhage, and lipid exudates. In such patients, neovascularization appears as a grayish discoloration in the macular area. Additional information on age-related macular degeneration (ARMD), including epidemiology, etiology/risk factors, clinical presentation, diagnosis, treatment and prevention is included in Jorge G. Arroyo, “Age-related macular degeneration-I and II,” UpToDate (on-line service) (last modified Sep. 19, 2005), which is hereby incorporated herein by reference.

In one embodiment, the compositions and methods of the invention provide for treating age-related macular degeneration or related disorders in subjects at risk for developing ARMD. A number of possible risk factors have been identified, of which age and smoking are the only factors that appear to definitively increase risk. Additional risk factors are set forth in Jorge G. Arroyo, “Age-related macular degeneration-I and II,” UpToDate (on-line service), as referenced above.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below. All references cited herein are hereby incorporated by reference in their entireties.

What is claimed:

1. A method of treating or preventing age-related macular degeneration (ARMD) in a subject in need thereof comprising administering to the subject a peptide/phospholipid complex wherein the peptide is the peptide of sequence SEQ ID NO:1.

2. The method of claim 1 wherein the phospholipid is selected from the group consisting of phosphatidylcholine, egg phosphatidylcholine, soybean phosphatidylcholine, dipalmitoylphosphatidylcholine, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, brain sphingomyelin, dipalmitoylsphingomyelin and distearoylsphingomyelin.

3. The method of claim 2 wherein the phospholipid is selected from the group consisting of dipalmitoylphosphatidylcholine, sphingomyelin or 1-palmitoyl-2-oleoylphosphatidylcholine.

4. The method of claim 1 wherein the peptide/phospholipid complex comprises a peptide of the sequence SEQ ID NO:1, sphingomyelin and 1,2-dipalmitoyl-sn-glycero-3-phosphocholine.

5. The method of claim 1 wherein the peptide/phospholipid complex consists essentially of a peptide of the sequence SEQ ID NO:1, sphingomyelin and 1,2-dipalmitoyl-sn-glycero-3-phosphocholine.

6. The method of claim 1, wherein the peptide/phospholipid complex is administered at an intravenous dose of about 1 mg/kg to about 20 mg/kg.

7. The method of claim 6, wherein the peptide/phospholipid complex is administered at an intravenous dose of about 5 mg/kg to about 10 mg/kg.

8. The method of claim 7, wherein the peptide/phospholipid complex is administered at an intravenous dose of about 5 mg/kg.

9. The method of claim 7, wherein the peptide/phospholipid complex is administered at an intravenous dose of about 10 mg/kg.

10. The method of claim 1, wherein the peptide/phospholipid complex is administered at an intra-ocular dose of about 0.2 to about 1 mg/kg.

11. The method of claim 10, wherein the peptide/phospholipid complex is administered at an intra-ocular dose of about 0.2 mg/kg.

12. The method of claim 10, wherein the peptide/phospholipid complex is administered at an intra-ocular dose of about 0.5 mg/kg.

13. The method of claim 10, wherein the peptide/phospholipid complex is administered at an intra-ocular dose of about 1 mg/kg.

14. The method of claim 1 wherein the ARMD is nonexudative or dry age-related macular degeneration.

15. The method of claim 1 wherein the ARMD is exudative or wet age-related macular degeneration.

16. The method of claim 1 wherein the complex is administered as a pharmaceutical formulation.

17. The method of claim 16 wherein the pharmaceutical formulation is a sterile liquid pharmaceutical formulation.
